(19) United States
(12) Patent Application Publication
(10) Pub. No.: US 2004/0015092 A1
(43) Pub. Date: Jan. 22, 2004

(54) Apparatus and method

(76) Inventor: Hans Pettersson, Linghem (SE)

Correspondence Address: YOUNG & THOMPSON, 745 SOUTH 23RD STREET 2ND FLOOR, ARLINGTON, VA, 22202

(21) Appl. No.: 10/296,171

(22) PCT Filed: May 15, 2001
(86) PCT No.: PCT/SE01/01065

(51) Int. Cl. A61B 5/097

(52) U.S. Cl. 600/532; 604/94.01

(57) ABSTRACT

A method and an apparatus for monitoring a patient's breath and supplying a gas or gases different from ambient air to the patient, includes: a manifold having a gas inlet for supplying the gas(es) to one of the patient's nostrils and a through passage for breathing air flowing solely to and from the patient's other nostril, and an optical sensor for sensing breathing air flowing in the through passage. It also refers to a nose adapter for use in the apparatus, the adapter including: the body forming a first through passage and a second through passage, the first and second passages extending through the body, respectively.

### FIELD OF THE INVENTION

The present invention concerns an apparatus and a method for monitoring a breathing air flow of a patient and supplying gas or gases different from ambient air and an adapter. In this application the term “patient” refers to a human being or an animal.

### BACKGROUND OF THE INVENTION

During clinical procedures there is a risk for respiratory arrest, apnoea, or changes in respiratory rate. This situation can for example result from administration of drugs, pain therapy or as a consequence of intoxication or trauma. Therefore, monitoring of breathing is an existing demand in such situations.

We have developed a reliable, real-time monitor and a method for monitoring breathing using an optical sensor. The monitor comprises means for transmitting light and measuring reflecting light and an optical sensor. The optical sensor comprises an optical fibre which at one end is connected to the monitor and at the other end is to be positioned in a breathing air flow of a patient. At the head of the fibre, the fibre end is cut preferably perpendicular to the extension of the fibre. Light is transmitted through the fibre which reflects at the flat end.

When a patient exhale, a humid air flow passes the head of the fibre and condences on the flat end. When the patient thereafter inhales, the condenced water on the lens evaporates due to differences in relative humidity. Different amounts of light is reflected by the flat end when it is dry and when it is wet. Therefore, a monitoring can be performed. This is what we call an optical sensor. For further details see our own patent EP 681 453, incorporated herein by reference.

To supply oxygen is a standard method for supporting the oxygen level in the blood of a patient and is applied in ambulances, emergency care, when a patient has repiratory problems and so on. Conventionally the oxygen is supplied by means of a tubing provided with two nasal cannulas for positioning in the nostrils of the patient.

In our earlier patent application WO 99/03395, incorporated herein by reference, an adapter, to be positioned in one of a patient's nostrils, provided with an optical sensor for monitoring breathing air flows from both the mouth and the nose is shown. In one example the adapter is “hooked” over a tubing provided with two nasal cannulas for oxygen supply, where the adapter replaces one of the nasal cannulas in the oxygen supply tubing and leads breathing air flows both from the mouth and nose to the optical sensor.

One of the problems with the above mentioned assembled device according to WO 99/03395 is that it is not suitable for babies and children under the age of about five years. Another problem is that it can be irritating having the adapter in the nostril since the adapter expands the nostril to be able to stay in place in the nostril. A further problem is that the adapter may move around and will then not be in the right position and therefore cause a hassle. Yet another problem is that the whole assembled device has to be thrown away after use.

### SUMMARY OF THE INVENTION

An aim of the present invention is to provide an apparatus that is easy and comfortable to use, which also can be used for babies and small children. Another aim is to provide an apparatus which is better for the environment. Further, there is an aim to be able to produce the apparatus according to the invention accurately with as little effort as possible.

According to a broad aspect of the invention it provides an apparatus for monitoring a patient's breath and supplying a gas or gases different than ambient air to the patient, comprising:

a manifold to be connected to the patient's nose and having a gas inlet for supplying the gas(es) to one of the patient's nostrils and a through passage for breathing air flowing solely to and from the patient's other nostril, and

an optical sensor for sensing breathing air flowing in said through passage.

According to a particular aspect of the invention it provides an apparatus for monitoring a patient's breath and supplying a gas or gases different than ambient air to the patient, comprising:

a manifold to be applied to the patient's nose and having a first through passage for communication with a first nostril of the patient, a second through passage for communication solely between the patient's second nostril and ambient air, and a gas inlet to said first through passage for supplying the gas(es) to the patient's first nostril via said first through passage, and

an optical sensor for sensing breathing air flowing in said second through passage.

According to another aspect of the invention it provides a method for monitoring a patient's breath and supplying a gas or gases different than ambient air to the patient, comprising:

supplying the gas(es) to one of the patient's nostrils; and

sensing by means of an optical sensor solely the patient's breathing air passing the patient's other nostril.

The invention also relates to a nose adapter for use in an apparatus for monitoring a patient's breath and supplying a gas or gases different than ambient air to the patient, the adapter comprising:

a body,

said body forming a first breathing passage and a second breathing passage, said first and second passages extending through said body, respectively.

An advantage with the method, apparatus and nose adapter according to the invention is that it suits everybody, from the smallest neonatal baby to a grown up. Another advantage is that it is comfortable to wear since the nostrils are not expanded by anything, the apparatus rests against the nose and has a light, slim embodiment. A further advantage is that the apparatus is easy to manufacture with high accuracy despite its small dimensions.

Preferably the gas inlet is directed substantially orthogonally to said first through passage of said manifold and said first through passage comprises preferably an enlarged chamber where said gas inlet opens into said first through passage. This gives the advantage that the gas(es) is able to fill upp the chamber thus letting the patient inhale a larger amount of gas(es).

To obtain the most reliable monitoring of the patient's breath a sensor head of the optical sensor is positioned in said second through passage of said manifold and directed substantially orthogonally to said second through passage. This may be done by placing the optical sensor in a recess inside the manifold or by attaching, for example by snap on means, the sensor to the manifold at the end of the second through passage, at the end facing away from the patient.

The apparatus may further comprise an adapter, which is releasably connected to said manifold for abutting the nose of the patient, but may also be designed without needing an adapter. An advantage of having an adapter according to the present invention is that it is better for the environment since only the adapter needs to be replaced for different patients.

One way of connecting the adapter to said manifold is by frictional engagement. Another solution-would for example be a snap on function. One advantage of frictional engagement is that it is easy and inexpensive to provide.

Said adapter and manifold are designed as matching male and female connection parts and preferably said manifold is designed as a female connection part and said adapter is designed as a male connection part fitting in said manifold.

Said first and second through passages further extend through said adapter, which preferably comprises two nostril protrusions to be inserted into the patient's nostrils, whereby said first and second through passages extend through said two nostril protrusions, respectively. The nostril protrusions may have any suitable shape, such as spherical, truncated cone arranged in either direction, mushroom or “power station”, i.e. having a waist. But the nostril protrusions are optional. If they are non existent the adapter will abut the nose of the patient without protruding into the nostrils of the patient.

The apparatus further comprises a tubing connected to said manifold for supplying the gas(es) and said optical sensor comprises an optical fibre connected to said manifold. The tubing and the optical fibre may be used to apply the apparatus on the patient.

In a first embodiment of said adapter the body thereof forms a gas inlet to said first through passages for supplying a gas or gases different than ambient air into said first through passage and forms a recess for receiving a sensor head of an optical sensor extending into said second through passage.

Each of the nostril protrusions of the adapter tapers from said body. This gives the advantage that the nostrils are sealed at the ends of the nostril protrusions closest to the body of the adapter but tapers so that the nostrils of the patient will not be expanded.

Said body and nostril protrusions are elastic and are preferably made of an elastic, injection moldable plastic. This gives-the advantage of a comfortable adapter which is easy to manufacture. In the case of an apparatus without a separate adapter, the manifold may at least partially be made of an elastic, injection mouldable plastic.

Preferably said first breathing passage is separate from said second breathing passage.

### BRIEF DESCRIPTION OF THE DRAWINGS

The present invention will now be described in detail in the following, taken in conjunction with the accompanying drawings illustrating by way of examples the principles of the invention.

FIG. 1 illustrates an embodiment of an apparatus according to the present invention provided with a gas tubing, an optical sensor and an adapter according to the present invention in perspective view.

FIG. 2 illustrates the apparatus in FIG. 1 in another perspective view.

FIG. 3 illustrates a first embodiment of a manifold according to the present invention from the side that is connectable to an adapter according to the present invention.

FIG. 4 illustrates the manifold in FIG. 3 in a sectional view.

FIG. 5 illustrates a portion of the manifold in FIG. 3.

FIG. 6 illustrates a first embodiment of an adapter according to the present invention in a side view.

FIG. 7 illustrates the side of the adapter in FIG. 6 that is connectable with the manifold according to the present invention.

FIG. 8 illustrates the first embodiment of the manifold and the adapter in FIG. 6 connected in a sectional view.

FIG. 9 illustrates a second embodiment of the manifold according to the present invention from the side that is connectable to an adapter according to the present invention.

FIG. 10a+b illustrates a third embodiment of the manifold in perspective and from above, respectively.

FIG. 11 illustrates an third embodiment of the adapter from the side that is connectable with the manifold.

FIG. 12 illustrates a fourth embodiment of the manifold from the side facing away from the patient and a connectable sensor.

FIG. 13 illustrates a fifth embodiment of the manifold from the side.

### DETAILED DESCRIPTION OF PREFERRED EMBODIMENTS OF THE PRESENT INVENTION

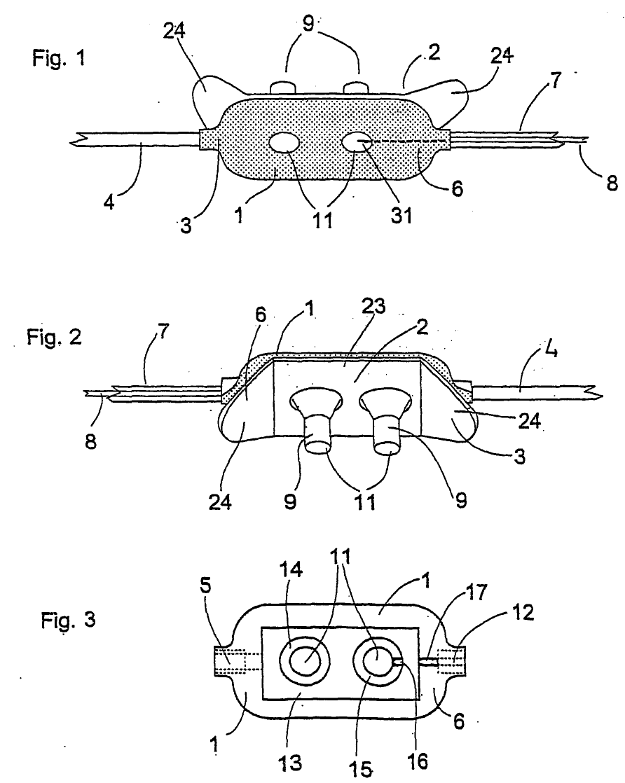

FIGS. 1 and 2 illustrate an apparatus according to the present invention comprising a manifold 1 provided with an adapter 2. The apparatus has two through passages 11 extending through the manifold 1 and the adapter 2. At a first end 3 of the manifold 1 a tubing 4 is connected to an inlet 5 for a gas or gases, for example oxygen, and at a second end 6 a tubing 7 is connected, in which tubing 7 an optical fibre 8 is provided.

The adapter 2 is provided with two nostril protrusions or nasal cannulas 9 intended to be positioned in the nostrils of a patient in which the through passages extend. Through the first nasal cannula 9 oxygen is supplied to the patient and in the second nasal cannula 9 the breathing air flow of the patient is lead to and through the manifold 1, in which the head 31 of an optical sensor is provided.

The manifold 1 with adapter 2 is positioned on the patient by means of the tubings 4 and 7 that pass around the head of the patient in opposite directions and meet at the back or at the neck where suitable connecting means is provided, such as a piece of larger tubing threaded onto both of the tubings keeping them in place by frictional forces.

In FIG. 3 a first embodiment of the manifold 1 is shown from the side which is connectable with an adapter 2. At the first end 3 the tubing 4 is attached in the inlet 5, for example by applying an adhesive, and at the other, second end 6 there is a conduit 12 for the optical fibre 8 of the optical sensor and its tubing 7 in which conduit 12 the tubing 7 is attached, for example by applying an adhesive.

On this side of the manifold 1 a recess 13 is provided, which may have any shape, in this case a rectangular shape. In the recess 13 the adapter 2 is intended to be positioned. Of course the opposite is possible, too, when the manifold has a protrusion that fits in a recess of the adapter. The two through passages 11 are provided at least partially in the manifold 2, orthogonally to the plane of the manifold 2.

The first through passage 11, provided closer to the first end 3 of the manifold 1, has only a slight protrusion 14 around its edge for sealing engagement with the adapter 2. The second through passage 11, provided closer to the second end 6 of the manifold 1, has a cylindrical protrusion 15 preferably extending to the same level as the side of the manifold 2. The cylindrical protrusion 15 is provided with a recess 16 for receiving the optical fibre 8.

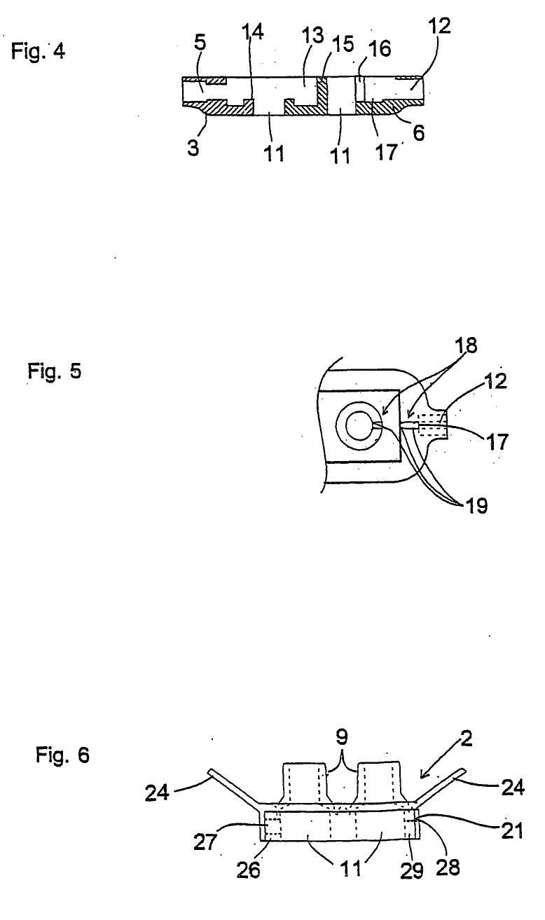

At the second end 6 of the manifold 1 a recess 17 is formed for holding the optical fibre 8, see FIGS. 3, 4 and 5. In the recess 17 and the recess 16 in the cylindrical protrusion 15 there are provided mechanical attachment means 18 formed like two opposite edges 19. The optical fibre 8 comprises the fibre embedded in a coating.

To attach the optical fibre 8 in the manifold I the optical fibre 8 is lead through the conduit 12, where the tubing 7 is attached, and further until the head of the fibre extends into the second through passage 11. Thereafter the optical fibre 8 is pressed down in the recesses 16 and 17 between the edges 19 so that the edges 19 deform the coating of the optical fibre 8 but not the fibre itself.

Preferably the manifold is manufactured by injection molding in a shape without sharp edges and a slim, curved outer side.

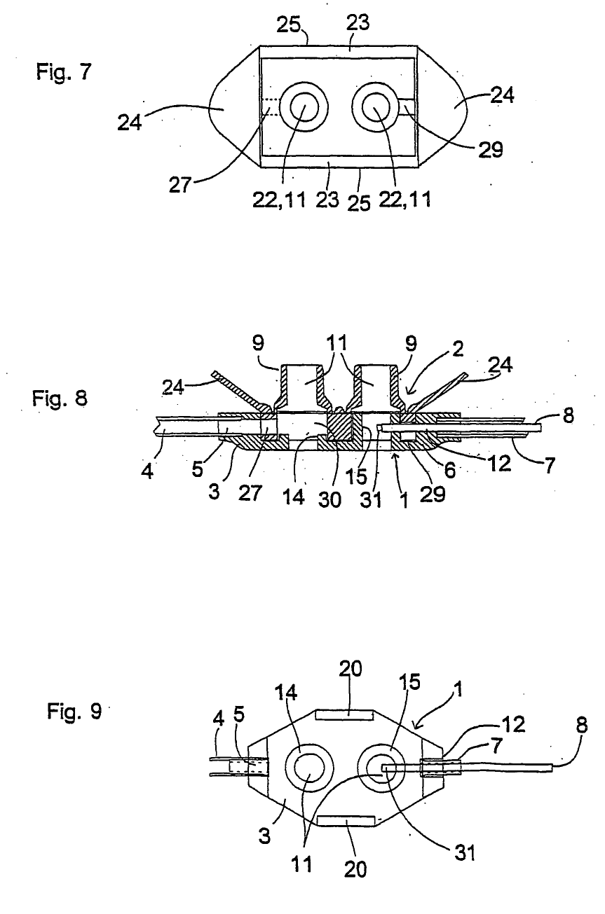

According to a first embodiment of an adapter 2, see FIGS. 6 and 7, it comprises a body 21 with an extension of the through passages 11 from the manifold 1 that starts as two holes 22 in the body 21 at the side of the adapter 2 that is connectable to the manifold 1 and then forms two nasal cannulas 9 at the opposite side of the body 21.

The nasal cannulas 9 are preferably conically tapered from the roots over at least a portion of the nasal cannulas 9. Thus the nasal cannulas 9 are thinner att the top and do not expand the nostrils of the patient but still seal at the bottom region of the cannulas 9 against the edges of the nostrils.

Around the edge of the body 21 at the side that is provided with the nasal cannulas 9 a flange 23 is provided. At the ends of the adapter 2 the flange 23 protrudes longer and slightly inclined towards the nasal cannulas 9 to form support flanges 24 that rest against the wings of the patient's nose when in use. The support flanges 24 are symmetrical so that the adapter may be used with either of its long sides 25 upwards when in use.

The portion of the body 21 that protrudes underneath the flange 23 is adapted to fit in the recess 13 in the manifold 1, i.e. its shape corresponds to the shape of the recess 13. In the shown case the body 21 is rectangular in shape and so is the shape of the recess 13 in the first embodiment of the manifold 1.

In the first end 26 of the adapter 2, corresponding to the first end 3 of the manifold, there is an inlet 27 for gas(es) in the portion of the body 21 between the first end 3 of the manifold 1 and the first through passage 11. The inlet 27 is aligned with the gas inlet 5 in the manifold 1 when the adapter 2 is positioned in the manifold 1.

In the opposite end, i.e. the second end 28, of the adapter 2 there is a recess 29 in the portion of the body 21 between the second end 6 of the manifold and the second through passage 11. The recess 29 is provided to receive the optical fiber 8 attached in the manifold 1 when the adapter 2 is positioned in the manifold 1.

As shown in the first embodiment of the manifold 1 the first through passage 11 is provided with only a slight protrusion 14 around the edge for sealing engagement with the adapter 2. Thus a larger chamber 30 is provided for the gas(es) in the first through passage 11 than if also this first channel were provided with a cylindrical protrusion 15 like the one at the second through passage 11, which is to be positioned inside the second hole 22 in the adapter 2, see FIG. 8.

A variant of the first embodiment of the adapter 2 does not have any inlet 27 for gas(es) but a recess to receive an extended inlet 5 of the other shown embodiment of the manifold 1. Otherwise it shows the features of the first embodiment of the adapter 2.

A second embodiment of the manifold 1 is shown in FIG. 9. The manifold 1 is shown from the side that is connectable with an adapter 2. The first end 3 of the manifold 1 has a gas inlet 5, in which the tubing 4 is attached, that extend all the way to a first cylindrical protrusion 14′. The second end 6 of the manifold 1 has a conduit 12 for the optical fibre 8, in which the tubing 7 for the optical fibre 8 is attached, that extends all the way to a second cylindrical protrusion 15′. The cylindrical protrusions 14′ and 15′ provide at least a portion of the first and second through passages 11.

A second embodiment of an adapter 2 may be connected to the second embodiment of the manifold 1, engaging frictionally over the inlet 5, conduit 12 and the cylindrical protrusions 14′ and 15′. Preferably some kind of additional support is provided at the manifold, too, in the shown case two sidewalls 20.

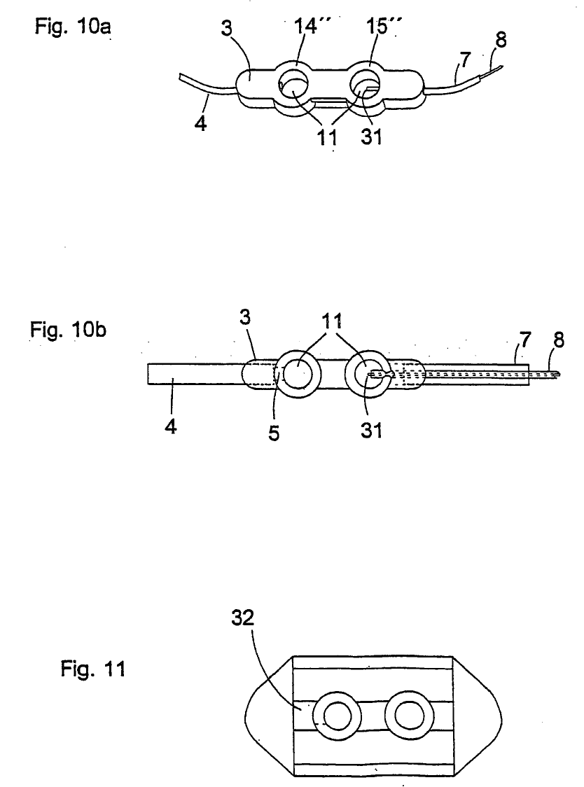

A third embodiment of the manifold 1 is illustrated in FIG. 10. This embodiment is especially suitable for neonatal babies and small babies. In the first end 3 the tubing 4 is provided in the inlet 5 for gas and in the second end 6 there is a conduit 12 for the optical fibre 8 and its tubing 7. The manifold 2 comprises two through passages 11 surrounded by cylindrical walls 14″ och 15″. This manifold 1 is to be inserted in an adapter 3 of a third embodiment, see FIG. 11, provided with a recess 32 for the manifold 1.

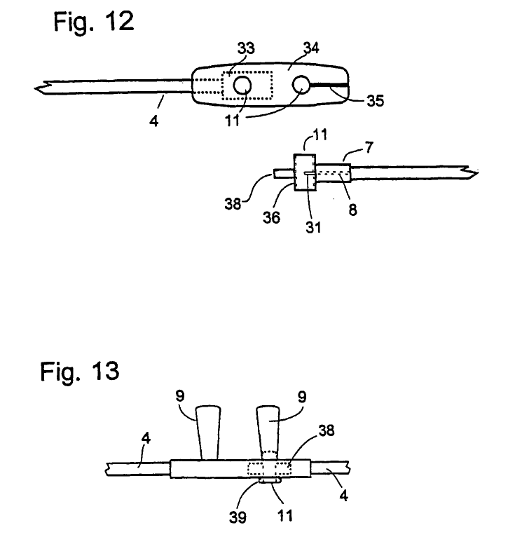

A fourth embodiment of the manifold is illustrated in FIG. 12. Said manifold comprises a first inner piece 33, an outer cover 34 and a connectable second inner piece 36. The first inner piece 33 is preferably made of a hard plastic and has a first through passage 11 and a gas inlet into the through passage connected to a gas supply tube 4. Opposite the gas inlet there may be some kind of attachment means 38 for attachment of the second inner piece 36 to the first inner piece 33.

The outer cover 34 is preferably made of a soft and elastic material and covers the first inner piece 33 and the end of the tubing 4 and has a space for receiving the second inner piece 36 and a slit 35 so that the second inner piece 36 may be positioned and taken out of the cover 34. The cover 34 may have nostril protrusions (not shown).

The second inner piece 36 is provided with a second through passage 11 and an optical sensor, whose head 31 is arranged in the second through passage 11 and the fibre 8 extends through a tubing 7 attached to the second inner piece 36. Opposite the optical sensor some kind of attachment means 38 is provided.

In FIG. 13 a fifth embodiment of the manifold is illustrated. This embodiment is an adapted conventional gas supply means with gas supply tubings 4 attached in each end and provided with two nostril protrusions 9. Opposite one of the nostril protrusions 9 a hole is made into which-an inner piece 38 is pushed. The inner piece 38 is provided with a through passage 11 and seals so that gas and breathing through the passage 11 will not mix. At the end of the through passage that is faced way from the patient, snap on means 39 is provided onto which an optical sensor may be attached.

The adapter 2 and/or parts of the manifold 1 is preferably manufactured of a soft and elastic material, most preferable of a plastic that is suitable for injection molding, for example Cawiton® from Wittenburg BV, The Netherlands.

Preferably the manifold is manufactured in three different sizes to suit babies, children and grown-ups. To each manifold size a range of adapters of different sizes and/or shapes, for example three sizes, may be provided to suit as many sizes and shapes of noses as possible without having to manufacture too many different parts.

1. An apparatus for monitoring a patient's breath and supplying a gas or gases different from ambient air to the patient, comprising:
a manifold to be applied to the patient's nose and having a first through passage for communication with a first nostril of the patient, a second through passage for communication solely between the patient's second nostril and ambient air, and a gas inlet to said first through passage for supplying the gas(es) to the patient's first nostril via said first through passage,
an inner piece providing a seal between said first and second through passage and comprising at least a portion of said second through passage,
an optical sensor for sensing breathing air flowing in said second through passage attached at the inner piece.

2. The apparatus according to claim 1, wherein said gas inlet is directed substantially orthogonally to said first through passage of said manifold.

3. The apparatus according to claim 1, wherein said first through passage comprises an enlarged chamber where said gas inlet opens into said first through passage.

4. The apparatus according to claim 1, wherein said optical sensor comprises a sensor head positioned in said second through passage of said manifold.

5. The apparatus according to claim 4, wherein said sensor head is directed substantially orthogonally to said second through passage of said manifold.

6. The apparatus according to claim 1, wherein said adapter is resilient.

7. The apparatus according to claim 1, wherein said adapter is removably connected to said manifold by frictional engagement thereto.

8. The apparatus according to claim 7, wherein said adapter and manifold are designed as matching male and female connection parts.

9. The apparatus according to claim 8, wherein said manifold is designed as a female connection part and said adapter is designed as a male connection part fitting on said manifold.

10. The apparatus according to claim 1, wherein said first and second through passages further extend through said adapter.

11. The apparatus according to claim 10, wherein said adapter comprises two nostril protrusions to be inserted into the patient's nostrils, said first and second through passages extending through said two nostril protrusions, respectively.

12. The apparatus according to claim 1, further comprising a tubing connected to said manifold for supplying the gas(es).

13. The apparatus according to claim 1, wherein said optical sensor comprises an optical fibre connected to said manifold.

14. A nose adapter releasably attached to an apparatus for monitoring a patient's breath and supplying a gas or gases different than ambient air to the patient, the adapter comprising:
a body,
said body forming a first through passage for communication with a first nostril of the patient, a second through passage for communication solely between the patient's second nostril and ambient air, said first and second passages extending through said body, respectively,
said body forms a recess for receiving a sensor head of an optical sensor extending into said second through passage, and
two nostril protrusions projecting from said body to be inserted into the patient's nostrils in which said first and second through passages extends, respectively.

15. The adapter according to claim 14, wherein said body forms a gas inlet to said first through passages for supplying a gas or gases different from ambient air into said first through passage.

16. The adapter according to claim 14, wherein each said nostril protrusion tapers from said body.

17. The adapter according to claim 15, wherein said body and nostril protrusions are resilient.

18. The adapter according to claim 15, wherein said first through passage is separate from said second through passage.

19. The adapter according to claim 14, wherein it is made of a resilient, injection mouldable plastic.

20. A method for monitoring a patient's breath and supplying a gas or gases different from ambient air to the patient, comprising:
supplying the gas(es) to one of the patient's nostrils; and
sensing by means of an optical sensor solely the patient's breathing air passing the patient's other nostril
by means of an apparatus according to claim 1 or claim 14.
